(19) United States
(12) Patent Application Publication
(10) Pub. No.: US 2006/0148707 A1
(43) Pub. Date: Jul. 6, 2006

(54) Use of ligands to GABA beta receptors

(76) Inventors: Raymond Bernasconi, Oberwil (CH); Uwe Otten, Basel (CH)

Correspondence Address: ROCCO ZANZONICO;33 SACHEM ROAD, LAKE HOPATCONG, NJ, 07849, US

(21) Appl. No.: 11/365,437

(22) Filed: Mar. 1, 2006

Related U.S. Application Data
(63) Continuation of application No. 09/955,381, filed on Sep. 18, 2001, now abandoned.
Application No. 09/955,381 is a continuation of application No. PCT/EP00/02605, filed on Mar. 23, 2000.

(30) Foreign Application Priority Data
Mar. 25, 1999 (GB) 9906882.7

(51) Int. Cl. A61K 38/17; A61K 31/19; A61K 31/195

(52) U.S. Cl. 514/12; 514/561; 514/557

(57) ABSTRACT

The invention relates to the use of ligands to GABA_{B} receptors for increasing neurotrophin levels in the central nervous system.

The present invention relates to a new pharmaceutical use of ligands to GABA_{B} receptors. Such compounds include GABA_{B} receptor antagonists, GABA_{B} receptor agonists, GABA_{B} receptor partial antagonists, GABA_{B} receptor partial agonists and allosteric modulators of GABA_{B} receptors.

More particularly the present invention relates to the use of ligands to GABA_{B} receptors for increasing neurotrophin levels in the central nervous system (CNS).

The neurotrophins are known to be involved in neuronal survival the growth and differentiation of synaptic efficacy and plasticity (Lo, D. C., 1996, Neuron 15, 979-981; Lessmnann V., 1998, Gen. Pharmac. 31, 667-674). For example, animal studies have shown that neurotrophins can reduce or prevent age-related axotomy or neurotoxin-induced neuronal loss or reduced function in a variety of brain regions and nerve growth factor (NGF) significantly improves cognition in aged rats (Fernandez C. et al., 1995, Mol. Chem. Neuropathol. 24, 241-244). Moreover, one case report has shown that the intracerebroventricular (i.c.v.) infusion of NGF in a patient with Alzheimer's disease (AD) resulted in an increase in nicotine binding in the frontal and temporal cortices and in a persistent increase in cortical blood flow combined with a significant improvement of verbal episodic memory (Olson L. et al., 1992, J. Neural Transm. [P-D Sect.] 4, 79-95). These preliminary results have recently been confirmed by Jönhagen et al. Dement. Geriatr. Cogn. Disord. 1998: 9, 246-257, who showed that i.c.v. infusion of NGF in three patients with AD for three months leads to improvement of CNS effects including upregulation of nicotinic receptors in the brain, an increased cortical blood flow, a decrease in slow-wave EEG activity and an improved performance in cognitive tests. The occurrence of two negative side effects (back pain and marked weight reduction) required to stop this clinical trial after three months. These reports suggested that NGF counteracts the cholinergic deficit in AD. The possibility that naturally occurring degeneration of the basal forebrain system, such as that seen in AD, may be inhibited by exogenous neurotrophin administration, opens up the field of neurotrophin therapy for neurodegenerative diseases. However, the clinical utility of neurotrophic factors is limited by the fact that they do not cross the blood-brain barrier and are easily metabolised by peptidases when administered peripherally (Barinaga, M. 1994, Science 264, 772-774). Thus, the need of invasive neurosurgical procedures (i.c.v. delivery catheter) severely restricts the utility of neurotrophins such as NGF and brain-derived neurotrophic factor (BDNF) as therapeutic agents for the treatment of behavioral and cognitive deficits related to aging and neurodegenerative diseases. Systemic treatment with trophic factors also causes serious side effects (Barinaga; see above).

Thus, there is a need to find means of stimulating endogenous neurotrophin synthesis in the brain by administration of substances that cross the blood brain barrier.

In accordance with the present invention it has now surprisingly been found that ligands to GABA_{B} receptors, particularly GABA_{B} receptor antagonists, enhance expression of neurotrophin mRNA and protein levels in various brain regions. Moreover it has been found that GABA_{B} receptor antagonists exert said activity with a remarkably long duration of action.

GABA_{B} antagonists are known for example from U.S. Pat. No. 5,051,524 or U.S. Pat. No. 5,332,729. Specific GABA_{B} antagonists include for example 3-{1(S)-[3-(cyclohexylmethyl)hydroxyphosphinyl)-2(S)-hydroxy-propylamino]ethyl}benzoic acid (hereinafter compound A), 3-(1(R)-[3-(cyclohexylmethyl)hydroxyphosphinyl-2(S)-hydroxy-propylamino]ethyl}benzoic acid (hereinafter compound B) and 3-aminopropyl-(n-butyl)-phosphinic acid, and their salts. For a review on GABA_{B} receptor antagonists and their therapeutic applications, see for example Bittiger, et al., TIPS 1993: 14, 391-393.

GABA_{B} agonists are known for example from U.S. Pat. No. 5,281,747. Specific GABA_{B} agonists include for example β-(aminomethyl)-4-chlorobenze propanoic acid (hereinafter compound C) and 3-amino-2S-hydroxypropyl methylphosphinic acid.

The effects of ligands to GABA_{B} receptors on the expression of neurotrophins is indicated in studies performed for example as follows:

Male adult Wistar rats (10-12 weeks) are killed by decapitation. The brains are removed, rapidly dissected with nuclease-free instruments on an ice-cold metal-plate, transferred to sterile cryotubes, and immediately shock-frozen by immersions in liquid nitrogen. Tissues are stored at −80° C. until further processing. Samples for the measurement of neurotrophin protein levels are prepared according to Lüesse et al., Exp. Brain Res. 1998: 119, 1-8. The homogenates are centrifuged at 12,500 g for 60 min, and the supernatants are used for NGF, BDNF and neurotrophin-3 (NT-3) quantification using specific ELISA assays.

Total cellular RNA is isolated according to the TRIZOL®-protocol (Gibco BRL, Eggenstein) as described by Lüesse et al. (see above).

Before being subjected to RT-PCR, the RNA extracts are supplemented with internal control RNA and optimized as described in detail by Heese K. et al., 1998, Neural Notes III, 21-23. Total RNA of each sample is first reverse-transcribed into cDNA which in turn is subjected to PCR amplification using primers specific for NGF and BDNF as described by Heese K et al., (see above).

For the quantification of neurotrophin transcripts, the ratios of densitometric scores for NGF or BDNF and S12 PCR products are calculated. Data are means±SEM of three independent experiments, each done in duplicate. N=4 to 6 for each group.

To measure the immunoreactive NGF into the cortex, hippocampus and spinal cord of rats, a two-site ELISA is used (Weskamnp, G. and Otten, U. 1987, J. Neurochem. 48, 1779-1786). Anti-β (2.5S, 7S) NGF and anti-β (2.5S, 7S) NGF-β-gal (clone 27/21) (Boehringer Mannheim) are applied, and the NGF content in the samples is determined by comparison with an NGF standard curve (absorbance measurement at 595 nm using an ELISA reader, Dynatech MR 700). For quantification of BDNF and NT-3 levels, specific immunoassay systems are used according to the manufacturer's (Promega) protocols but modified by Heese, K. and Otten, U., 1998, J. Neurochem. 70, 699-707. Statistical evaluation of results is performed by applying analysis of variance, and the statistical error is the SEM. Recovery is 80% using recombinant mouse NGF as internal standard.

In these tests, GABA_{B} receptor antagonists at doses of about 0.1 to about 600 mg/kg i.p. significantly increase NGF- and BDNF-mRNA in the cortex, hippocampus and spinal cord 6 to 24 hours after treatment and significantly increase NGF- and BDNF-protein levels in said regions 12 to 72 hours after treatment.

For example with compound B on administration of 1 and 6 mg/kg i.p., a 3 to 4-fold increase of NGF mRNA and a 2.0 to 4.0-fold increase of BDNF mRNA is induced in said regions 6 and 24 hours after treatment, whereas with compound A on administration of 3 and 10 mg/kg i.p., a 2.0 to 2.5-fold increase of NGF mRNA and a 2 to 3-fold increase of BDNF mRNA is induced in said regions 6 and 24 hours after treatment. Similarly with compound B, on administration of 1 mg/kg i.p., a 1.5 to 2-fold increase of NGF protein (peak values at 24-48 hours after treatment) and a 2 to 2.5-fold increase of BDNF protein (peak values at 72 hours) is induced in said regions.

In the same tests, GABA_{B} receptor agonists at doses of about 0.1 to about 10 mg/kg i.p. significantly increase NT-3-protein levels in the cortex, hippocampus and spinal cord 24 to 96 hours after treatment.

For example with compound C on administration of 4 mg/kg i.p., a 3-fold increase of NT-3-protein levels is induced in said regions.

Ligands to GABA_{B} receptors are therefore useful in the treatment of any condition responsive to an increase of neurotrophin levels in the CNS. Particularly GABA_{B} receptor antagonists are useful for the treatment of conditions responsive to an increase of NGF and BDNF, and GABA_{B} receptor agonists are useful for the treatment of conditions responsive to an increase of NT-3.

Such conditions include neurodegenerative diseases such as Alzheimer's and related diseases, and stress-induced neurodegeneration; motor neuron diseases, e.g. amyotrophic lateral sclerosis, spinal muscular atrophy and post-polio syndrome; Parkinson's disease and syndromes, and suppression of immune responses following CNS tissue grafts; Huntington's chorea and other basal ganglia disorders; spinal cord injury and head trauma; neuroinflammation, e.g. multiple sclerosis, inflammatory hyperalgesia; severe depression states; schizophrenia; furthermore peripheral neuropathy; convulsive states, e.g. status epilepticus and excitotoxic/ischemic damages.

For the above-mentioned indications the appropriate dosage will of course vary depending upon, for example, the compound employed, the host, the mode of administration and the nature and severity of the condition being treated. However, in general, satisfactory results in animals are indicated to be obtained at a daily dosage of from about 0.1 to about 600 mg/kg body weight. In larger mammals, for example humans, an indicated daily dosage is in the range from about 1 to about 2000 mg of a compound for use according to the invention conveniently administered, for, example, in divided doses up to five times a day.

The present invention accordingly provides the use of a ligand to GABA_{B} receptors in the treatment of the above-mentioned conditions.

For use according to the invention, the ligand to GABA_{B} receptors may be administered as single active agent or in combination with other active agents, in any usual manner, e.g. orally, for example in the form of tablets or capsules, or parenterally, for example in the form of injection solutions or suspensions.

Moreover, the present invention provides pharmaceutical compositions comprising the ligand to GABA_{B} receptors in association with at least one pharmaceutical carrier or diluent for use in the treatment of any of the above-indicated diseases. Such compositions may be manufactured in conventional manner. Unit dosage forms may contain, for example, from about 0.25 to about 500 mg of the ligand to GABA_{B} receptors.

The present invention also provides the use of a ligand to GABA_{B} receptors for the manufacture of a pharmaceutical composition for the treatment of any of the above-indicated diseases.

The invention furthermore provides a method for increasing neurotrophin levels in the CNS, particularly for the treatment of any of the above-indicated diseases, in a subject in need of such treatment, which comprises administering to said subject a therapeutically effective amount of a ligand to GABA_{B} receptors.

1.-3. (canceled)

4. A method for increasing neurotrophin levels in the in the central nervous system (CNS) of a patient with Parkinson's disease, amyotrophic lateral sclerosis, or stress-induced neurodegeneration, said method comprising administering to the patient an amount of a GABA_{B} receptor antagonist sufficient to increase neurotrophin levels in the CNS of the patient.

5.-11. (canceled)

12. A method for increasing neurotrophin levels in the central nervous system (CNS) of a patient with Parkinson's disease, comprising administering to the patient an amount of a GABA_{B} receptor antagonist sufficient to increase neurotrophin levels in the CNS of a patient with Parkinson's disease.

13. A method for treating Parkinson's disease, comprising administering to a patient in need of such treatment a therapeutically effective amount of a GABA_{B} receptor antagonist, thereby treating the patient.

14. The method of claim 12 wherein the antagonist is administered daily.

15. The method of claim 13 wherein the antagonist is administered daily.

16. The method of claim 4 wherein the GABA_{B} receptor antagonist is selected from the group consisting of 3-{1(S)-[3-(cyclohexylmethyl)hydroxyphosphinyl)-2(S)-hydroxy-propylamino]ethyl}benzoic acid; 3-{1(R)-[3-(cyclohexylmethyl)hydroxyphosphinyl-2(S)-hydroxy-propylamino]ethyl}benzoic acid; and 3-aminopropyl-(n-butyl)-phosphinic acid.

17. The method of claim 12 wherein the GABA_{B} receptor antagonist is selected from the group consisting of 3-{1(S)-[3-(cyclohexylmethyl)hydroxyphosphinyl)-2(S)-hydroxy-propylamino]ethyl}benzoic acid; 3-{1(R)-[3-(cyclohexylmethyl)hydroxyphosphinyl-2(S)-hydroxy-propylamino]ethyl}benzoic acid; and 3-aminopropyl-(n-butyl)-phosphinic acid.

18. The method of claim 13 wherein the GABA_{B} receptor antagonist is selected from the group consisting of 3-{1(S)-[3-(cyclohexylmethyl)hydrokyphosphinyl)-2(S)-hydroxy-propylamino]ethyl}benzoic acid; 3-{1(R)-[3-(cyclohexylmethyl)hydroxyphosphinyl-2(S)-hydroxy-propylamino]ethyl}benzoic acid; and 3-aminopropyl-(n-butyl)-phosphinic acid.

19. The method of claim 4 where the GABA_{B} receptor antagonist is administered to a patient with Parkinson's disease or amyotrophic lateral sclerosis.

20. The method of claim 19, wherein the GABA_{B} receptor antagonist is administered to a patient with Parkinson's disease.
